(19) United States
(12) Patent Application Publication
(10) Pub. No.: US 2007/0032782 A1
(43) Pub. Date: Feb. 8, 2007

(54) Method and apparatus for eye alignment

(76) Inventors: Gerhard Youssefi, Landshut (DE); Friedrich Moritz, Munich (DE)

Correspondence Address: Bausch & Lomb Incorporated, One Bausch & Lomb Place, Rochester, NY, 14604-2701, US

(21) Appl. No.: 10/549,220

(22) PCT Filed: Feb. 18, 2004
(86) PCT No.: PCT/EP04/01555

§ 371 Date: May 31, 2006

(30) Foreign Application Priority Data
Mar. 24, 2003 (DE) 103 13 028.4

(51) Int. Cl. A61F 9/008; A61B 18/18

(52) U.S. Cl. 606/11; 606/5

(57) ABSTRACT

In an ophthalmic laser system preferably intended for photoablative refractive surgery, a component apparatus that is preferably an optical coherence tomography device for measuring corneal pachymetry makes its measurement when the First and Second Purkinje reflections of the OCT probe beam are detected, otherwise, the reflection signal is not strong enough to enable the OCT measurement. The beam axis of the therapeutic laser of the system is co-aligned with the OCT-prbbe beam. When the First and Second Purkinje reflections of the OCT probe beam are detected, a signal is generated by the OCT device and sent to the eye tracker component of the system to engage the eye tracker operation. This allows for objective, automatic engagement of the eye tracker and alignment of the patient's optical axis to the treatment axis or a diagnostic beam axis.

### BACKGROUND OF THE INVENTION

1. Field of the Invention

The invention is generally directed to the field of refractive surgery and is more particularly directed to a system, apparatus, and method for eye alignment and eyetracker engagement.

2. Description of Related Art

Extreme accuracy is typically required whenever surgery is performed. This requirement is underscored when the surgery is performed on a part of the body that is subject to involuntary movement. In the preferred field of the instant invention, refractive surgery is performed on a patient's eye in a common procedure known as LASIK, for example, or similar procedures such as PRK or LASEK. In all of these cases, a laser beam typically having a wavelength of 193 nm is used to photoablate volumetric portions of an exposed corneal surface to provide a new shape to the corneal surface for correction of visual defects.

In general, it is a problem to align the patient's eye. The eye is subject to saccades which are quick, involuntary movements of small magnitude. A person may voluntary shift their gaze during surgery; and furthermore, eye position stability is affected by the patient's heartbeat and other physiological factors. Moreover, there is still debate over what is the proper reference axis for alignment of the eye for laser refractive surgery. Some surgeons, for example, prefer to identify the center of the pupil, however, pupil center location is pupil-size dependent. Some surgeons use the Purkinje axis of the eye to align the eye at the therapeutic system. This can be problematic because the Purkinje axis is characterized by having an overlap of several reflexes of an illumination laser beam from the cornea. For a more detailed description of alignment axes, the interested reader is directed to Uozato and Guvton, American Journal of Ophthalmology, 103: 264-275, March 1987, which is hereby incorporated by reference in its entirety to the fullest allowed extent.

In typical laser ophthalmic systems for correction of refractive defects, an eyetracker component of the system is utilized to track the motion of the eye during surgery, and to interrupt delivery of the therapeutic laser beam when tracking cannot be maintained. Various eye tracker technologies are commercially available and are not, per se, germane to the invention described herein below. It is however necessary to engage the eye tracker when it is locked onto the desired reference point on the eye. Often, the surgeon will engage the eye tracker manually when it “looks” to be properly aligned. This subjective technique is prone to error which may lead to decentered ablations and other impediments to satisfactory vision correction. Accordingly, the inventors have recognized a need for more reliability and accuracy in eye alignment, particularly as it applies to successful laser ophthalmic surgery.

### SUMMARY OF THE INVENTION

In accordance with an embodiment of the invention, an ophthalmic laser surgery system including a therapeutic laser that outputs a beam along a beam axis and an eye tracker, incorporates a cooperating component that emits a probe beam having an optical axis which is co-aligned and concentric with the therapeutic beam axis and which emits a signal upon detection of a First Purkinje reflex of the probe beam and a Second Purkinje reflex of the probe beam when the First and Second Purkinje reflections are co-aligned and concentric. The signal is then used to trigger operation of the eye tracker.

In another embodiment, an eye tracker system that monitors the movement of a patient's eye during an ophthalmic procedure can be automatically engaged upon receiving a signal emitted by a cooperative, separate, diagnostic component that functions by suitably detecting at least two different reflections of a probe beam from the cornea, when the component detects a concentric co-alignment of a First Purkinje reflex and a Second Purkinje reflex of the probe beam from the patient's eye.

Another embodiment of the invention is directed to a method for aligning an optical axis of a patient's eye with a therapeutic axis of an ophthalmic therapeutic apparatus and/or a diagnostic axis of an ophthalmic diagnostic apparatus, and includes the steps of directing a probe beam having a propagation axis that is co-aligned and concentric with the therapeutic axis and/or diagnostic axis onto the eye, detecting a First Purkinje reflex of the probe beam, detecting a Second Purkinje reflex of the probe beam, and upon detecting a concentric co-alignment of the First and Second Purkinje reflections from the eye, establishing the alignment of the patient's optical axis with the therapeutic axis and/or diagnostic axis. In an aspect of this embodiment, a further step includes generating a signal upon detection of the concentric co-alignment of the First and Second Purkinje reflections. In a further aspect, the method includes using the signal to engage an eye tracker device that is in cooperative engagement with the ophthalmic therapeutic apparatus and/or the ophthalmic diagnostic apparatus.

Another embodiment is directed to an ophthalmic system for measuring and/or correcting a vision defect of a patient's eye, including a diagnostic component for measuring the vision defect or, preferably, a therapeutic component for correcting the vision defect, and an eye tracking component in cooperative engagement with the diagnostic component and/or the therapeutic component for monitoring the movement of the eye in regard to the measurement and/or correction of the vision defect, wherein engaging the eye tracking component when the optical axis of the patient's eye is aligned with a beam axis of the diagnostic component and/or the therapeutic component is accomplished by providing a device component in cooperative engagement with the system that emits a probe beam into the eye having an optical axis that is co-aligned and concentric with the beam axis of the diagnostic component and/or the therapeutic component, detecting a First Purkinje reflex of the probe beam and a Second Purkinje reflex of the probe beam when the First and Second Purkinje reflections are co-aligned and concentric, and generating a signal upon the detection, and using the signal to trigger operation of the eye tracking component.

In all of the foregoing embodiments, an optical coherence tomography (OCT) device is the preferable component and means for generating the probe beam, detecting the Purkinje reflections, and generating the signal for triggering the eye tracker.

These and other objects of the present invention will become more readily apparent from the detailed description to follow. However, it should be understood that the detailed description and specific examples, while indicating the preferred embodiments of the invention, are given by way of illustration only, since various changes and modifications within the spirit and scope of the invention will become apparent to those skilled in the art based upon the description and drawings herein and the appended claims.

### BRIEF DESCRIPTION OF THE DRAWINGS

FIG. 1 is a schematic illustration of a cross-section of an eye;

FIG. 2 is a schematic illustration of a system embodiment according to the invention; and

FIG. 3 is a graphical illustration of a triggering signal according to an embodiment of the invention.

### DETAILED DESCRIPTION OF PREFERRED EMBODIMENT OF THE INVENTION

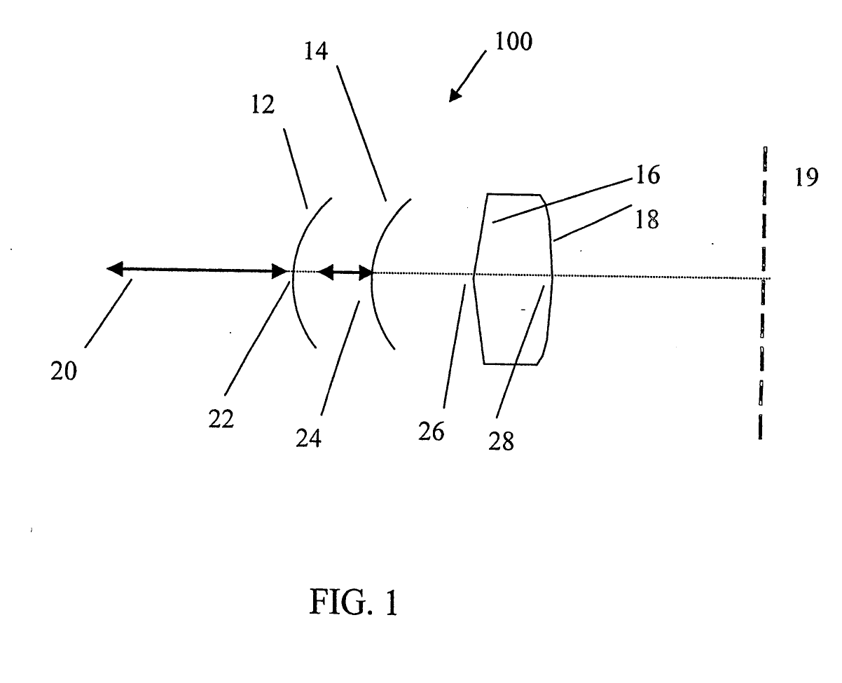

The invention is directed to apparatus and methods for objectively aligning the optical axis of a patient's eye with the beam axis of a diagnostic or a therapeutic component, for example, an excimer laser, of a refractive vision correction surgery system. An embodiment of the invention is further directed to the automatic engagement or triggering of an eyetracker in such a system. The invention is based on the detection of the co-alignment of First and Second Purkinje reflections from the patient's eye, as illustrated in FIG. 1. In FIG. 1, a cross-section of the eye 100 is shown schematically to include an anterior corneal surface 12, a posterior corneal surface 14, an anterior lens surface 16, a posterior lens surface 18, and a retinal surface 19 (shown as a straight, dashed line for illustration purposes). It has long been understood by persons skilled in the art that when an eye is appropriately illuminated by an input beam 20, four Purkinje reflections can be detected. The First Purkinje reflex 22 is defined as the virtual image formed by the light reflected from the anterior surface of the cornea 12. The Second Purkinje reflex 24 is an image of the input light formed by the reflection from the posterior corneal surface 14. The light that is not reflected from either the anterior corneal surface or the posterior corneal surface propagates through the cornea and the aqueous humor, and through the lens of the eye onto the retina 19. The Third Purkinje reflex 26 is a virtual image formed by the input light 20 reflected from the anterior surface of the eye lens 16, while the Fourth Purkinje image is formed by light reflected from the posterior surface of the lens 18 at its interface with the vitreous humor. The interested reader is directed to P. N. Cornsweet and H. D. Crane, J. Opt. Soc. Am., 63, 921 (1973) for a more detailed discussion of Purkinje image formation, which reference is herein incorporated by reference in its entirety.

Ocular pachymetry, particularly corneal pachymetry (corneal thickness measurement), is a valuable measurement parameter in surgical ophthalmic procedures such as refractive vision correction, for example. Several techniques have been developed to measure corneal pachymetry including, for example, ultrasonic measurements and optical coherence tomography (OCT).

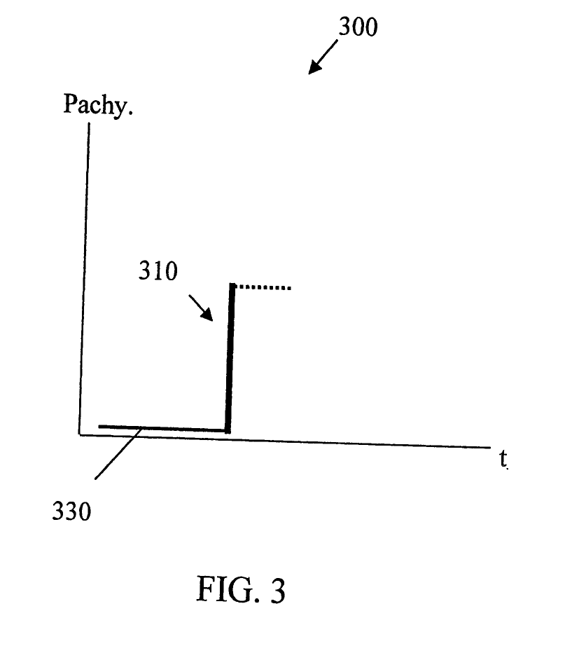

The principles of OCT are familiar to those skilled in the art and for the purpose of the present invention encompass optical coherence reflectometry and other forms of optical interferometry that can be used to obtain corneal thickness measurements. The interested reader is directed to Hitzenberger, “Measurement of Corneal Thickness by Low Coherence Interferometry”, Applied Optics, Vol. 31, No. 31 (November 1992) which is herein incorporated by reference in its entirety to the extent allowed by applicable laws and rules. In essence, a signal from an OCT apparatus is generated only when the beam path of the OCT probe radiation reflected from a measurement surface is equal to a reference beam path established in the OCT apparatus to within a distance corresponding to the temporal coherence length of the OCT radiation. In order to measure the central thickness of the cornea the OCT device must recognize the reflection of the probe beam from the anterior corneal surface 12 corresponding to the First Purkinje reflex 22, and reflection from the posterior corneal surface 14 corresponding to the Second Purkinje reflex 24. As shown in FIG. 3, the pachymetry signal 330 is essentially zero until the coincident reflection of the First and Second Purkinje images are detected at 310. At this point, the corneal pachymetry has been measured by the apparatus and, according to the invention, this signal can be used to trigger an eyetracker apparatus for monitoring the movement of the eye during a diagnostic or therapeutic procedure or other eye tracker function. In a conventional eyetracker system, the patient may be asked to fixate on an illumination source while a visible laser beam coincident with a therapeutic beam axis is directed onto the patient's cornea. Based upon the surgeon's observation of the visible laser beam in relation to the corneal position, the surgeon will manually engage the eyetracker using his or her best judgment about the corneal position. Advantageously, according to the invention, the eyetracker can now be triggered automatically and more accurately since the OCT signal will only be generated when the patient's optical axis is properly aligned.

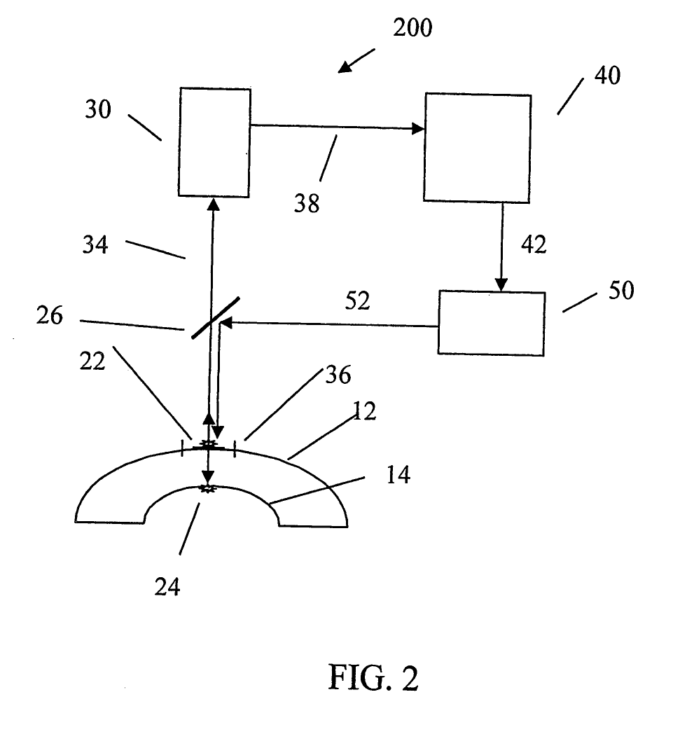

A system embodiment of the invention is shown schematically in FIG. 2. The system 200 represents a photoablative eye surgery system for reshaping a patient's cornea represented by anterior corneal surface 12 and posterior corneal surface 14. The system includes an OCT component 30 that emits a probe beam 34 which passes through beam splitter 26 and propagates towards the eye. The beam is apertured by known aperturing means 36 to preferably restrict the probe beam diameter to between about 200 to 300 microns. This is advantageous in that it restricts the probe beam scan over a small lateral dimension resulting in faster detection of the OCT signal. The system further includes a therapeutic laser component 50 that emits a therapeutic beam having a propagation axis as shown at 52. The probe beam 34 from OCT component 30 is co-aligned and coincident with therapeutic beam axis 52 at the corneal surface. The location of therapeutic beam axis 52 on the corneal surface during the therapeutic procedure is controlled by eyetracker 40 in a manner well known to those skilled in the art; that is, the motion of the eye due to voluntary and involuntary movement is monitored in real time to coordinate the ablation of the cornea with the therapeutic beam. According to the invention, a central corneal thickness measurement will be obtained by the OCT component 30 upon detection of the probe beam reflection 22 from the anterior corneal surface 12 and the probe beam reflection 24 from the posterior corneal surface 14 when these two reflections are co-aligned. Reflections 22 and 24 represent the First and Second Purkinje reflections, respectively. At the instant of successful corneal pachymetry measurement, the measurement signal received by the OCT component 30 is provided at 38 to the eyetracker 40 which is triggered by the signal 38 and communicated to the laser 50 by signal 42.

While various advantageous embodiments have been chosen to illustrate the invention, it will be understood by those skilled in the art that changes and modifications can be made therein without departing from the scope of the invention as defined in the appended claims.

1. In a laser eye surgery system including a laser apparatus for generating a therapeutic laser beam having a therapeutic beam axis and an eye tracker having an alignment axis for monitoring movements of the eye, wherein said eye tracker operates in cooperative engagement with said laser apparatus,
an improvement characterized by:
an apparatus in cooperative engagement with said laser eye surgery system adapted to emit a probe beam having an optical axis co-aligned and concentric with the therapeutic beam axis and adapted to emit a signal upon detection by said apparatus of a First Purkinje reflex of the probe beam and a Second Purkinje reflex of the probe beam when said First and Second Purkinje reflections are co-aligned and concentric, wherein said signal is used to trigger operation of the eye tracker.

2. The system of claim 1, further comprising a component having an aperture that limits the size of a region on an anterior surface of the cornea where the probe beam can intersect the cornea.

3. The system of claim 2, wherein the aperture has a diameter of between about 200 to 300 μm.

4. The system of any of claims 1 to 3, wherein the apparatus is an OCT device.

5. The system of any of claims 1 to 4, wherein the probe beam is in the IR region of the spectrum.

6. The system of any of claims 1 to 5, wherein the probe beam has a coherence length of between about 5 to 8 μm.

7. An ophthalmic surgery system, comprising:
a therapeutic laser component that provides a beam having a therapeutic beam axis;
an eye tracker component that monitors the motion of a patient's eye for locating the therapeutic beam on the eye; and
a device component that provides a probe beam having an optical axis that is co-aligned and concentric with the therapeutic beam axis, which can emit a signal upon detection by said device component of a First Purkinje reflex of the probe beam and a Second Purkinje reflex of the probe beam when said First and Second Purkinje reflections are co-aligned and concentric, wherein said signal is used to trigger operation of the eye tracker.

8. The system of claim 7, wherein the device component is an OCT device.

9. The system of claim 7 or 8, wherein the probe beam is in the IR region of the spectrum.

10. The system of any of claims 7 to 9, wherein the probe beam has a coherence length of between about 5 to 81 μm.

11. The system of any of claims 7 to 10, wherein the device component has an aperture that limits the size of a region on an anterior surface of the cornea where the probe beam can intersect the cornea.

12. The system of claim 11, wherein the aperture has a diameter of between about 200 to 300 μm.

13. An eye tracker system that monitors the movement of a patient's eye during an ophthalmic procedure, wherein said eye tracker system is automatically engaged upon receiving a signal emitted by a cooperative, separate, diagnostic component when said component detects a concentric co-alignment of a First Purkinje reflex and a Second Purkinje reflex of a beam directed onto the patient's eye.

14. The system of claim 13, wherein the cooperative component is an OCT device.

15. The system of claim 14, wherein the beam is emitted from the OCT device, said beam having a coherence length of between about 5 to 8 μm.

16. A method for aligning an optical axis of a patient's eye with a therapeutic axis of an ophthalmic therapeutic apparatus and/or a diagnostic axis of an ophthalmic diagnostic apparatus, comprising:
directing a probe beam having a propagation axis that is co-aligned and concentric with the therapeutic axis and/or diagnostic axis onto the eye;
detecting a First Purkinje reflex of the probe beam;
detecting a Second Purkinje reflex of the probe beam; and
detecting a concentric co-alignment of the First and Second Purkinje reflections from the eye, wherein said concentric co-alignment established the alignment of the patient's optical axis with the therapeutic axis and/or diagnostic axis.

17. The method of claim 16, comprising generating said probe beam from an OCT device.

18. The method of claim 17, comprising aperturing said probe beam to a diameter of between about 200 to 300 μm on an anterior surface of the patient's eye.

19. The method of claim 17 or 18, comprising generating said probe beam having a coherence length of between about 5 to 8 μm.

20. The method of any of claims 16 to 19, further comprising generating a signal upon detection of the concentric co-alignment of the First and Second Purkinje reflections.

21. The method of claim 20, comprising using the signal to engage an eye tracker device that is in cooperative engagement with the ophthalmic therapeutic apparatus and/or the ophthalmic diagnostic apparatus.

22. In an ophthalmic system for measuring and/or correcting a vision defect of a patient's eye, including at least one of a diagnostic component for measuring the vision defect and a therapeutic component for correcting the vision defect, and including an eye tracking component in cooperative engagement with the diagnostic component and/or the therapeutic component for monitoring the movement of the eye in regard to the measurement and/or correction of the vision defect, a method for engaging the eye tracking component when the optical axis of the patient's eye is aligned with a beam axis of the diagnostic component and/or the therapeutic component, comprising:
providing a device component in cooperative engagement with the system that emits a probe beam into the eye having an optical axis that is co-aligned and concentric with the beam axis of the diagnostic component and/or the therapeutic component;
detecting with said device component a First Purkinje reflex of the probe beam and a Second Purkinje reflex of the probe beam when said First and Second Purkinje reflections are co-aligned and concentric; and
generating a signal upon said detecting, and using said signal to trigger operation of the eye tracking component.

23. The method of claim 22, comprising providing an OCT device for generating the probe beam.

24. The method of claim 22 or 23, comprising aperturing said probe beam to a diameter of between about 200 to 300 μm on an anterior surface of the patient's eye.

25. The method of any of claims 22 to 24, comprising generating said probe beam having a coherence length of between about 5 to 8 μm.
